(19) United States
(12) Patent Application Publication
(10) Pub. No.: US 2022/0121700 A1
(43) Pub. Date: Apr. 21, 2022

(54) PICTURE-BASED MESSAGE GENERATION AND RETRIEVAL

(71) Applicant: MOTOROLA SOLUTIONS, INC., CHICAGO, IL (US)

(72) Inventors: CHIN CHUAN YAP, BAYAN LEPAS (MY); KAI BOON KHOO, AYER ITAM (MY); SYALWANI BINTI KAMARUDIN, KOTA BHARU (MY); BRIAN J. FROMMELT, DEER PARK, IL (US)

(21) Appl. No.: 17/074,660

(22) Filed: Oct. 20, 2020

(51) Int. Cl. G06F 16/532; H04W 4/90; G06F 16/535; G06V 40/16; G16H 30/20; G06F 16/51; H04W 88/02

(52) U.S. Cl. CPC G06F 16/532; H04W 4/90; G06F 16/535; H04W 88/02; G16H 30/20; G06F 16/51; G06V 40/172

(57) ABSTRACT

A method and apparatus for generating and retrieving information that is associated with a victim is provided herein. During operation, a medic will provide a picture of a victim's face to a server along with the information associated with the diagnosis and/or treatment of the victim. At a future time, if a second medic wishes to retrieve the information associated with the victim, the second medic will again provide a picture of the victim's face to the server. In response, the server will provide the information to the second medic.

### BACKGROUND OF THE INVENTION

During mass-casualty events, oftentimes there exist a limited number of doctors and paramedics on scene to treat the injured. This often-time results in doctors/paramedics leaving hand-written notes on victims after assessment and treatment so that other doctors/paramedics need not repeat the assessment and treatment of the same victim. Writing notes and somehow attaching them to a victim can take time, and is often difficult to do. It would be beneficial if an easier, more-reliable way to leave notes about a victim could be implemented.

### BRIEF DESCRIPTION OF THE SEVERAL VIEWS OF THE DRAWINGS

In the accompanying figures similar or the same reference numerals may be repeated to indicate corresponding or analogous elements. These figures, together with the detailed description, below are incorporated in and form part of the specification and serve to further illustrate various embodiments of concepts that include the claimed invention, and to explain various principles and advantages of those embodiments.

FIG. 1 shows a general operating environment for the present invention.

FIG. 2 illustrates associating notes with an image.

FIG. 3 is a block diagram of a note server of FIG. 1.

FIG. 4 illustrates the message flow between a device and a server.

FIG. 5 illustrates a message flow between a device and a server.

FIG. 6 is a flow chart showing operation of the note server of FIG. 3.

Skilled artisans will appreciate that elements in the figures are illustrated for simplicity and clarity and have not necessarily been drawn to scale. For example, the dimensions of some of the elements in the figures may be exaggerated relative to other elements to help improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

In order to address the above, mentioned need, a method and apparatus for generating and retrieving information that is associated with a victim is provided herein. During operation, a medic will provide a picture of a victim's face to a server along with the information associated with the diagnosis and/or treatment of the victim. At a future time, if a second medic wishes to retrieve the information associated with the victim, the second medic will again provide a picture of the victim's face to the server. In response, the server will provide the information to the second medic.

Expanding on the above, a server will receive a request from a first user to associate an image of a person's face (taken by the first user) with information provided by the first user. The server will store the image of the person's face along with the information. When a request is received at the server by a second user wishing to access the information, the request will be accompanied by another image of the persons face (taken by the second user). The server will attempt to match the image of the person taken by the second user to faces it has in storage. If a match can be made, the information associated with the matched person is provided to the second user. As is evident, the first user, the second user, and the person are different people.

For example, consider a medic at a mass-casualty scene. The medic assesses a person as being in stable condition with a possible broken leg. The medic takes a picture of the person's face and sends the picture along with the assessment to the server, and then moves on to treat other individuals. At a later time, paramedics arrive on scene to transport the person to the hospital. The paramedic can obtain the medic's assessment by simply taking a picture of the person's face and providing it to the server with a request for the assessment. In response, the server will match the two images of the persons face (i.e., identify the two images as being of the same person) and then provide the assessment to the paramedics.

Example embodiments are herein described with reference to flowchart illustrations and/or block diagrams of methods, apparatus (systems) and computer program products according to example embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. The methods and processes set forth herein need not, in some embodiments, be performed in the exact sequence as shown and likewise various blocks may be performed in parallel rather than in sequence. Accordingly, the elements of methods and processes are referred to herein as “blocks” rather than “steps.”

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the flowchart and/or block diagram block or blocks.

The computer program instructions may also be loaded onto a computer or other programmable data processing apparatus to cause a series of operational blocks to be performed on the computer or other programmable apparatus to produce a computer implemented process such that the instructions which execute on the computer or other programmable apparatus provide blocks for implementing the functions/acts specified in the flowchart and/or block diagram block or blocks. It is contemplated that any part of any aspect or embodiment discussed in this specification can be implemented or combined with any part of any other aspect or embodiment discussed in this specification.

Further advantages and features consistent with this disclosure will be set forth in the following detailed description, with reference to the figures.

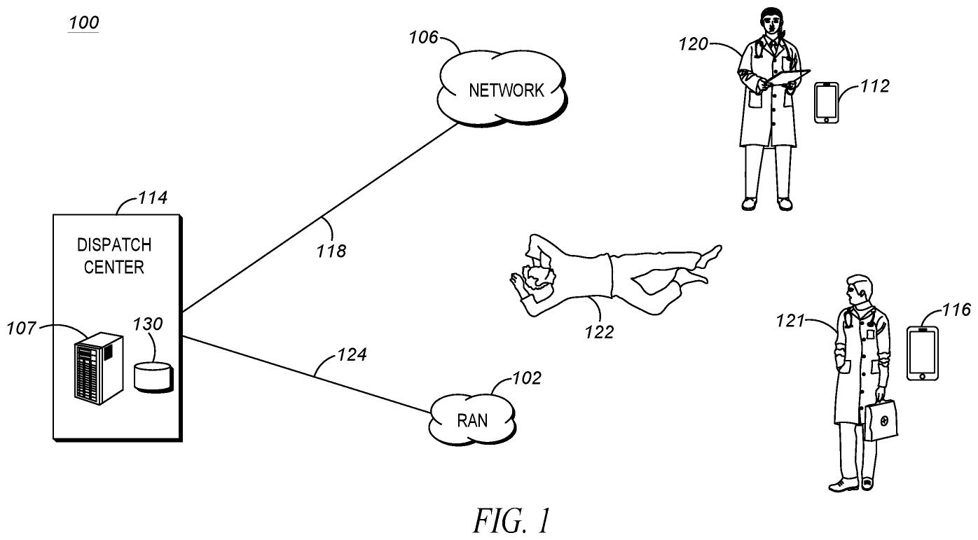

Turing now to the drawings wherein like numerals designate like components, FIG. 1 is a general operating environment 100 for the present invention. Environment 100 includes one or more devices 112 and 116, victim 122, doctors 120 and 121, dispatch center 114, and communication links 118, 124. Server 107 exists within dispatch center 114, and is configured to receive an image of a face and information regarding a victim's 122 medical condition. Server 107 is also configured to associate the image with the information, and store (in database 130) the image along with information. Server 107 is also configured to receive a request for information along with an image of person's 122 face. In response, server 107 will attempt to match the image of the person's face to any facial images in database 130. If a match is made, server 107 is configured to provide the information to the requester for the person whose image is matched.

As shown, server 107 is coupled to database 130. Database 130 comprises images along with associated medical information. It should be noted that although only one server 107 is shown coupled to database 130, there may exist many servers 107 providing above-described services doctors, paramedics, medics, and others.

In one embodiment of the present invention, server 107 exists within dispatch center 114. However, server 107 may exist separated from dispatch center 114. Additionally, shown in FIG. 1, two separate networks exist, radio-access network 102 and public network 106 (e.g., Verizon, Spring, AT&T, . . . , etc.). In alternate embodiments of the present invention more or fewer networks may be utilized to transmit images and information. Referring to the configuration shown in FIG. 1, RAN 102 includes typical RAN elements such as base stations, base station controllers (BSCs), routers, switches, and the like, arranged, connected, and programmed to provide wireless service to user devices 112 and 116 (e.g., a smart phone, a radio, a tablet computer, or any other smart device operated by individuals 120 and 121) in a manner known to those of skill in the relevant art.

In a similar manner, network 106 includes elements such as base stations, base station controllers (BSCs), routers, switches, and the like, arranged, connected, and programmed to provide wireless service to user devices in a manner known to those of skill in the relevant art.

Although not shown, a public-safety core network may exist between ran 102 and dispatch center 114 which includes one or more packet-switched networks and/or one or more circuit-switched networks, and in general provides one or more public-safety agencies with any necessary computing and communication needs, transmitting any data and communications.

Device 112 and device 116 may be any suitable computing and communication devices configured to engage wireless communication over an air interface as is known to those in the relevant art. Device 112 and device 116 may comprise any device capable of acquiring an image of a person and providing medical information to server 107. Thus, devices 112 and 116 comprise at least a camera (not shown) and means to send text, voice, or other data describing a medical condition to server 107. For example, devices 112 and 116 may comprise a mobile device such as a smart phone, police radio, tablet, computer, . . . , etc. running an application to perform the above features on an Android™ or iOS™ operating system and having a camera.

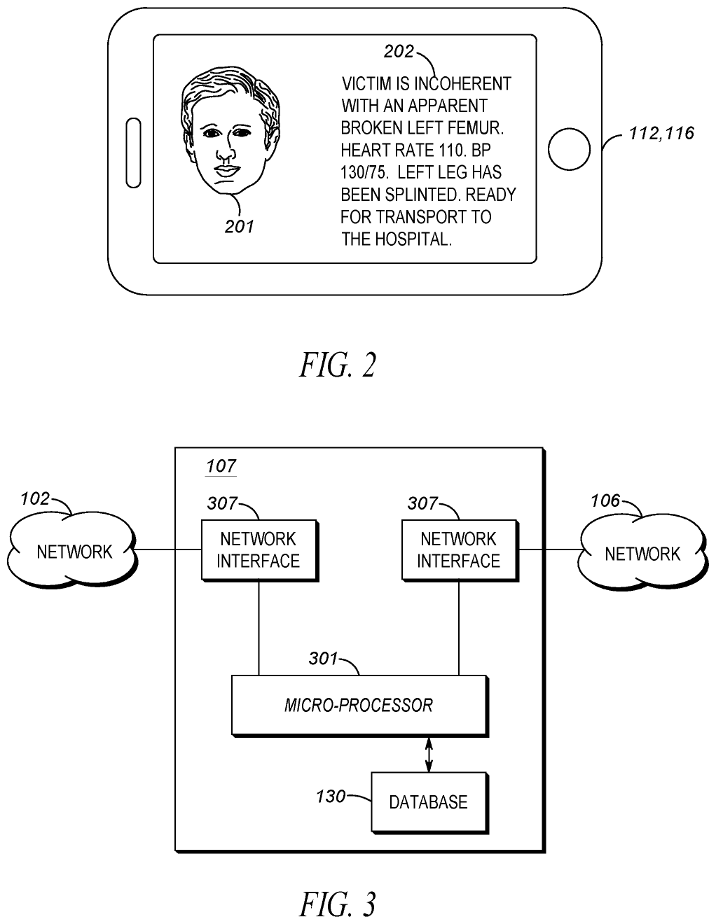

During operation, device 112 and 116 are configured (for example by running an application) to store information within server 107 regarding victim 122 by providing an image of the victim along with information regarding the victim's medical status to server 107. Devices 112 and 116 operated by users 120 and 121 respectively, are also configured to retrieve the information regarding the victim's medical status by sending a request for the information along with an image of the victim. This is illustrated in FIG. 2, where device 112, 116 has captured an image 201 of a face of patient 122 and has recorded medical information 202 about the victim. This information can be sent to server 107 for later retrieval.

FIG. 3 is a block diagram of server 107. In this embodiment server 107 has database 130 existing within server 107, however in alternate embodiments, database 130 may exist external to server 107. Regardless, server 107 includes network interfaces 307 and logic circuitry 301. In other implementations, server 107 may include more, fewer, or different components.

Network interfaces 307 may comprise a transmitter and receiver for wireless communications, and may be long-range and/or short-range transceivers that utilize a private 802.11 network set up by a building operator, a next-generation cellular communications network operated by a cellular service provider, or any public-safety network such as an APCO 25 network or the FirstNet broadband network. In addition, network interfaces may comprise network interface 106 and 102, elements including processing, modulating, demodulating, and transceiver elements that are operable in accordance with any one or more standard or proprietary wireless interfaces, wherein some of the functionality of the processing, modulating, and transceiver elements may be performed by means of the processing device 301 through programmed logic such as software applications or firmware stored on the storage component 130 (standard random access memory) or through hardware. Examples of network interfaces (wired or wireless) include Ethernet, T1, USB interfaces, IEEE 802.11b, IEEE 802.11g, etc.

Logic circuitry 301 comprises a digital signal processor (DSP), general purpose microprocessor, a programmable logic device, or application specific integrated circuit (ASIC) and is configured to store and provide medical information as described above. Logic circuitry 301 is also configured to perform facial recognition on any received image of a person to determine if the person has an additional image stored in memory 130.

Memory 130 comprises standard random-access memory, and is used to store medical information and associated facial images.

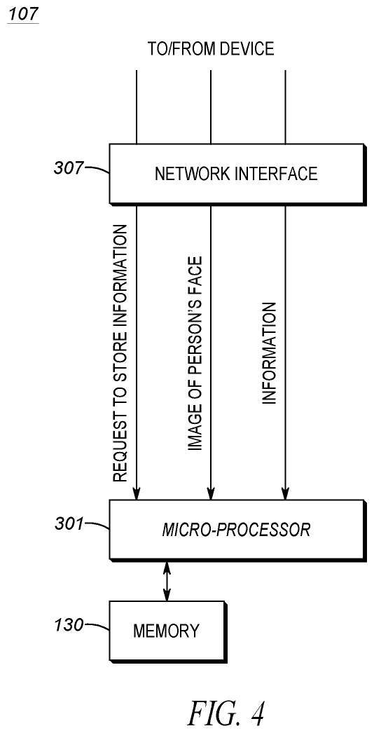

FIG. 4 is a block diagram of server 107 shown in FIG. 1. More specifically, FIG. 4 illustrates the message flow between server 107 and the various devices 112, 116 during storage of information. As is evident, a request to store information about a victim is sent from device 112, 116 to logic circuitry 301. This request may be in the form of a standard message sent between the device and server 107. Along with the request, an image of a person and information is provided by device 112, 116. In response, logic circuitry 301 associates the information with the image of the person and stores the image of the person and associated information.

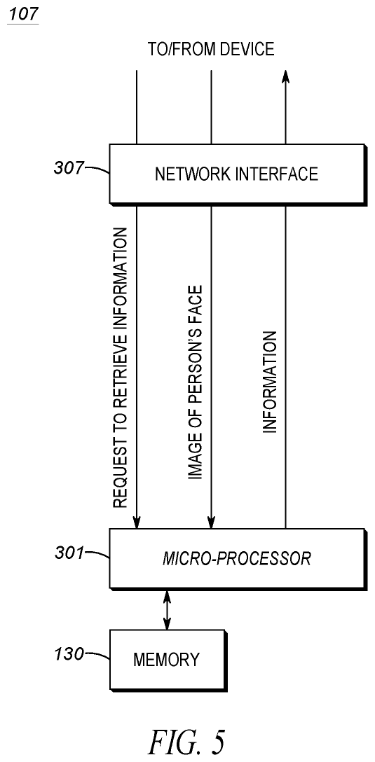

FIG. 5 is a block diagram of server 107 shown in FIG. 1. More specifically, FIG. 5 illustrates the message flow between server 107 and the various devices 112, 116 during retrieval of any victim information. As is evident, a request to retrieve information about a victim is sent from device 112, 116 to logic circuitry 301. This request may be in the form of a standard message sent between the device and server 107. Along with the request, an image of a person is provided by device 112, 116. In response, logic circuitry 301 performs facial recognition on the image of the person to see if that person has another image stored in memory 130. If so, then the information is provided to the device 112, 116.

Thus, server 107 comprises an apparatus comprising a network interface configured to receive a first image of a person's face at a first time from a first device operated by a first user. The network interface also configured to receive information from the first device regarding the person's medical status. The network interface also configured to receive a second image of a person's face at a second time from a second device, wherein the first image and the second image differ. Logic circuitry is provided, and configured to determine that the second image of the person's face and the first image of the person's face comprise images of a same individual, and provide the information from the first device to the second device when it is determined that the second image of the face and the first image of the face comprise different images of the same individual.

As discussed above, the first and second devices comprise wireless devices. Additionally, a database is provided and the logic circuitry is also configured to associate the information from the first device with the first image of the face by storing the information from the first device along with the first image of the face within the database. Finally, the logic circuitry is configured to perform facial recognition on the second image of the face to determine that the second image of the face and the first image of the face comprise different images of the same individual.

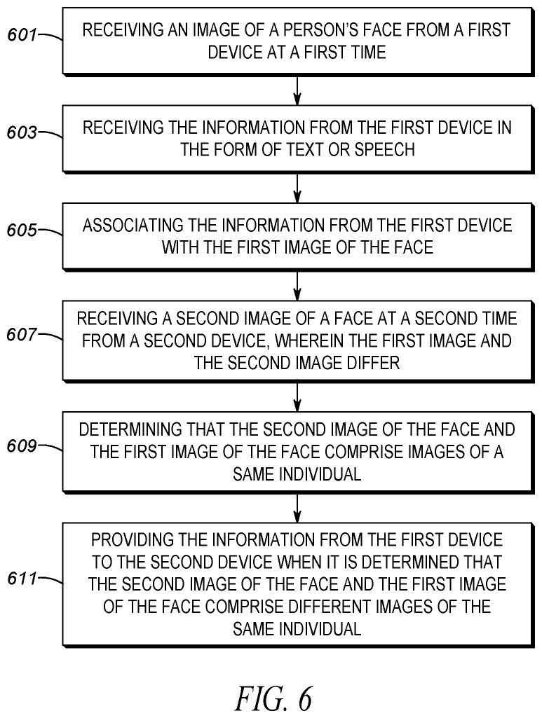

FIG. 6 is a flow chart showing operation of server 107. The logic flow begins at step 601 where logic circuitry 301 receives an image of a face of person 122 from a first device 112 at a first time. At step 603, logic circuitry 301 receives information from the first device in the form of text or speech. More particularly, a simple text message may be provided, or a recording of a person's voice may be provided.

At step 605, logic circuitry 301 associates the information from the first device with the first image of the face, and at step 607 receives a second image of a face at a second time from a second device 116, wherein the first image and the second image differ. The logic flow continues to step 609 where logic circuitry performs facial recognition to determine that the second image of the face and the first image of the face comprise images of a same individual and provides the information from the first device to the second device when it is determined that the second image of the face and the first image of the face comprise different images of the same individual (step 611).

It is envisioned that the information is provided to the second device that exists at a public-safety incident scene

It should be noted that if a user request information about a victim, but provides an image that cannot be matched with anyone currently stored in database 130, logic circuitry will send a message back to the user telling the user that no match could be made with anyone currently on file. No information will be provided to the user.

As discussed above, the information comprises medical information about the person. Additionally, the first device is operated by a first user, and the person and the first user are different people. Both the first and the second device comprise a wireless device. Finally, the step of associating the information from the first device with the first image of the face comprises the step of storing the information from the first device along with the first image of the face within a database.

As discussed above, the step of determining that the second image of the face and the first image of the face comprise images of the same individual comprises the step of performing facial recognition on the second image of the face to determine that the second image of the face and the first image of the face comprise different images of the same individual.

In the foregoing specification, specific embodiments have been described. However, one of ordinary skill in the art appreciates that various modifications and changes can be made without departing from the scope of the invention as set forth in the claims below. For example, instead of facial recognition, any form of appearance matching may be used to identify victims. For example, analytics could use clothing to identify a victim. Additionally, while the above technique was applied to victims, one of ordinary skill in the art could apply the above technique to other scenarios such as customers, clients, patients in a clinic, or other scenarios where a first user may have an initial interaction with the person and a second user may have a related interaction with the same person in which notes from the first user would be of benefit to both the person and second user during their interaction. Accordingly, the specification and figures are to be regarded in an illustrative rather than a restrictive sense, and all such modifications are intended to be included within the scope of present teachings. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “one of”, without a more limiting modifier such as “only one of”, and when applied herein to two or more subsequently defined options such as “one of A and B” should be construed to mean an existence of any one of the options in the list alone (e.g., A alone or B alone) or any combination of two or more of the options in the list (e.g., A and B together).

A device or structure that is “configured” in a certain way is configured in at least that way, but may also be configured in ways that are not listed.

The terms “coupled”, “coupling” or “connected” as used herein can have several different meanings depending in the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through an intermediate elements or devices via an electrical element, electrical signal or a mechanical element depending on the particular context.

It will be appreciated that some embodiments may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Any suitable computer-usable or computer readable medium may be utilized. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. In the context of this document, a computer-usable or computer-readable medium may be any medium that can contain, store, communicate, propagate, or transport the program for use by or in connection with the instruction execution system, apparatus, or device.

Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. For example, computer program code for carrying out operations of various example embodiments may be written in an object oriented programming language such as Java, Smalltalk, C++, Python, or the like. However, the computer program code for carrying out operations of various example embodiments may also be written in conventional procedural programming languages, such as the “C” programming language or similar programming languages. The program code may execute entirely on a computer, partly on the computer, as a stand-alone software package, partly on the computer and partly on a remote computer or server or entirely on the remote computer or server. In the latter scenario, the remote computer or server may be connected to the computer through a local area network (LAN) or a wide area network (WAN), or the connection may be made to an external computer (for example, through the Internet using an Internet Service Provider).

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

What is claimed is:

1. A method for providing information to a user, the method comprising the steps of:
receiving a first image of a person's face from a first device at a first time;
receiving the information from the first device in a form of text or speech;
associating the information from the first device with the first image of the face;
receiving a second image of a face at a second time from a second device, wherein the first image and the second image differ;
determining that the second image of the face and the first image of the face comprise images of a same individual; and
providing the information from the first device to the second device when it is determined that the second image of the face and the first image of the face comprise different images of the same individual.

2. The method of claim 1 wherein the information comprises medical information about the person.

3. The method of claim 1 wherein the first device is operated by a first user, and the person and the first user are different people.

4. The method of claim 1 wherein the first device comprises a wireless device.

5. The method of claim 1 wherein the step of associating the information from the first device with the first image of the face comprises the step of storing the information from the first device along with the first image of the face within a database.

6. The method of claim 1 wherein the second device comprises a wireless device.

7. The method of claim 1 wherein the step of determining that the second image of the face and the first image of the face comprise images of the same individual comprises the step of performing facial recognition on the second image of the face to determine that the second image of the face and the first image of the face comprise different images of the same individual.

8. The method of claim 1 wherein the step of providing the information to the second device comprises the step of wirelessly transmitting the information to the second device at a public-safety incident scene.

9. A method for providing information to a device, the method comprising the steps of:
receiving a first image of a person's face at a first time from a first device that is operated by a first user, and wherein the first user and the person are different people;
receiving the information from the first device in a form of text or speech;
associating the information from the first device with the first image of the face;
receiving a second image of a face at a second time from a second device, wherein the first image and the second image differ;
determining that the second image of the face and the first image of the face comprise images of a same individual;
providing the information from the first device to the second device when it is determined that the second image of the face and the first image of the face comprise different images of the same individual;
wherein the first and second device comprise wireless devices;
wherein the step of associating the information from the first device with the first image of the face comprises the step of storing the information from the first device along with the first image of the face within a database,
wherein the step of determining that the second image of the face and the first image of the face comprise images of the same individual comprises the step of performing facial recognition on the second image of the face to determine that the second image of the face and the first image of the face comprise different images of the same individual; and
wherein the step of providing the information to the second device comprises the step of transmitting the information to the second device at a public-safety incident scene.

10. An apparatus comprising:
a network interface configured to receive a first image of a person's face at a first time from a first device operated by a first user, the network interface also configured to receive information from the first device regarding the person's medical status, the network interface also configured to receive a second image of a person's face at a second time from a second device, wherein the first image and the second image differ;
logic circuitry configured to:
determine that the second image of the person's face and the first image of the person's face comprise images of a same individual; and
provide the information from the first device to the second device when it is determined that the second image of the face and the first image of the face comprise different images of the same individual.

11. The apparatus of claim 10 wherein the first device comprises a wireless device.

12. The apparatus of claim 10 further comprising a database, and wherein the logic circuitry is also configured to associate the information from the first device with the first image of the face by storing the information from the first device along with the first image of the face within the database.

13. The apparatus of claim 10 wherein the second device comprises a wireless device.

14. The apparatus of claim 10 wherein the logic circuitry is configured to perform facial recognition on the second image of the face to determine that the second image of the face and the first image of the face comprise different images of the same individual.
